(19) United States
(12) Patent Application Publication
(10) Pub. No.: US 2014/0228548 A1
(43) Pub. Date: Aug. 14, 2014

(54) VIRAL CLEARANCE METHODS

(71) Applicant: Olga Galperina, North Potomac, MD (US)

(72) Inventor: Olga Galperina, North Potomac, MD (US)

(73) Assignee: Human Genome Sciences, Wilmington, DE (US)

(21) Appl. No.: 14/342,412

(22) PCT Filed: Aug. 31, 2012
(86) PCT No.: PCT/US2012/053313

§ 371 Date: Mar. 3, 2014

Related U.S. Application Data
(60) Provisional application No. 61/530,132, filed on Sep. 1, 2011.

(51) Int. Cl. C07K 1/22

(52) U.S. Cl. CPC C07K 1/22
USPC 530/413; 530/415

(57) ABSTRACT

The invention provides methods for separating a polypeptide of interest (such as an antibody) from a virus. In some embodiments, the methods involve eluting the polypeptide of interest from a Protein A resin with an elution buffer have a particular range of conductivity values that minimizes the amount of virus that co-elutes with the polypeptide of interest.

### FIELD OF THE INVENTION

The present invention relates to methods of purifying polypeptides using Protein A Chromatography to enhance viral clearance.

### BACKGROUND OF THE INVENTION

Polypeptides of interest, such as antibodies, are often produced in live cells. A cell line that expresses a polypeptide of interest must be sustained through complex media. Thus, purification of a polypeptide of interest includes the separation of the polypeptide from elements of the media, cellular components and other byproducts of the cell line.

In particular, it is imperative that the biotechnology industry consider viral contamination when polypeptides are produced from animal or human cell lines. Viruses can be present in the source material, introduced by the polypeptide production process or found in the growth media (Valdes, R. et al., J. Biotechnol. 96(3):251-8 (2002)). It is critical that viral impurities are removed or inactivated from the final biological products (U.S. Department of Health and Human Services, Guidance for industry: Q5A Viral safety evaluation of biotechnology products derived from cell line of human or animal origin. 1998). Thus, improved methods are needed to remove contaminating viruses from polypeptide products, such as therapeutic polypeptides.

### SUMMARY OF THE INVENTION

In general, the invention provides methods for removing contaminating virus from a polypeptide of interest using Protein A chromatography. In one such aspect, the invention provides a method for separating a polypeptide of interest from a virus. In some embodiments, the method involves applying an elution buffer having a conductivity between about 3.0 to about 10 mS/cm to a Protein A resin having a polypeptide of interest and a virus adsorbed to the resin. In some embodiments, the method involves applying an elution buffer comprising sulfate (such as about 50 or about 100 mM sodium sulfate) to a Protein A resin having a polypeptide of interest and a virus adsorbed to the resin. In some embodiments, the elution of the polypeptide of interest from the resin separates the polypeptide of interest from at least a portion of the virus.

In one aspect, the invention provides a method for purifying a polypeptide of interest. In some embodiments, the method involves applying a solution comprising the polypeptide of interest and a virus to a Protein A resin under conditions such that the polypeptide of interest binds to the Protein A resin. In some embodiments, the resin is washed with a wash buffer. In some embodiments, the wash step elutes one or more contaminants from the resin. In some embodiments, this wash step is omitted. In some embodiments, the polypeptide of interest is eluted from the resin with an elution buffer having a conductivity between about 3.0 to about 10 mS/cm to provide a recovered composition. In some embodiments, the polypeptide of interest is eluted from the resin with an elution buffer comprising sulfate (such as about 50 or about 100 mM sodium sulfate) to provide a recovered composition.

In one aspect, the invention features another method for purifying a polypeptide of interest. In some embodiments, the method involves applying a solution comprising the polypeptide of interest and a virus to the Protein A resin under conditions such that the polypeptide of interest binds to the Protein A resin. In some embodiments, the resin is washed with a wash buffer. In some embodiments, the wash step elutes one or more contaminants from the resin. In some embodiments, this wash step is omitted. In some embodiments, the polypeptide of interest is eluted from the resin with a first elution buffer to provide a recovered composition. In some embodiments, the amount of virus in the recovered composition is measured. In some embodiments, if the measured amount of virus is greater than desired, the method is repeated with a second elution buffer with a higher conductivity than the first elution buffer. In some embodiments, if the measured amount of virus is greater than desired, the method is repeated with a second elution buffer with more sulfate (such as sodium sulfate) than the first elution buffer. In some embodiments, the method is repeated using the recovered composition from the first cycle of the method to increase the purity of the recovered composition. In some embodiments, the method is repeated using a solution comprising the polypeptide of interest that has not been subjected to Protein A purification. This solution may be the same as or different from the solution purified during the first cycle of the method.

In some embodiments of any of the aspects of the invention, the amount of virus in the recovered composition is at least about any of 10, 10², 10³, 10⁴, 10⁵, or 10⁶-fold less than the amount of virus in the solution applied to the resin. In some embodiments, the amount of virus in the recovered composition is between about 10² to about 10⁶-fold less than the amount of virus in the solution applied to the resin, such as about 10³ to about 10⁶-fold, about 10⁴ to about 10⁶-fold, or about 10⁴ to about 10⁵-fold less. In some embodiments, the amount of two or more viruses is reduced.

In some embodiments of any of the aspects of the invention, the conductivity of the elution buffer is between about 3 to about 3.5, about 3.5 to about 4, about 4 to about 4.5, about 4.5 to about 5, about 5 to about 5.5, about 5.5 to about 6, about 6 to about 6.5, about 6.5 to about 7, about 7 to about 7.5, about 7.5 to about 8, about 8 to about 8.5, about 8.5 to about 9, about 9 to about 9.5, or about 9.5 to about 10 mS/cm. In some embodiments, the conductivity of the elution buffer is between about 3.0 to about 10 mS/cm, such as about 3.5 to about 9.5, about 4 to about 7, or about 5 to about 6 mS/cm. In some embodiments, the elution buffer comprises sodium sulfate, such as about 50 or about 100 mM sodium sulfate. In some embodiments, the elution buffer comprises sodium citrate, such as about any of 15, 20, or 25 mM sodium citrate. In preferred embodiments, the elution buffer comprises sodium citrate and sodium sulfate. In some embodiments, the pH of the elution buffer is between about 2 to about 5.5, such as about 2.5 to about 4.5, or about 3 to about 4. In some embodiments, the pH of the elution buffer is between about 2 to about 2.5, about 2.5 to about 3, about 3.0 to about 3.5, about 3.5 to about 4, about 4 to about 4.5, about 4.5 to about 5, or about 5 to about 5.5.

In some embodiments of any of the aspects of the invention, the polypeptide of interest comprises an antibody, antibody fragment, or a fusion polypeptide comprising an antibody or antibody fragment. In some embodiments, a virus is adsorbed to the Protein A resin, such as a virus that interacts with the Protein A or solid support portion of the resin, or a virus bound to the Protein A or solid support. In some embodiments, the virus is a virus that infects mammalian cells, such as cells used to produce the polypeptide of interest. In some embodiments, the virus is a retrovirus or single-stranded DNA virus. In some embodiments, the virus is a parvovirus.

It is to be understood that one, some, or all of the properties of the various embodiments described herein may be combined to form other embodiments of the present invention. These and other aspects of the invention are described in further detail below.

### BRIEF DESCRIPTION OF THE FIGURES

FIG. 1A shows the clearance of Murine Minute Virus (MMV) in the Protein A elution pool using different elution buffer conductivities.

FIG. 1B shows the clearance of Xenotropic Murine Leukemia Virus (XMuLV) in the Protein A elution pool using varying elution buffer conductivities.

### DETAILED DESCRIPTION OF THE INVENTION

The present invention is directed to methods of purifying a polypeptide of interest using Protein A chromatography to enhance viral clearance. Viruses can potentially be introduced into the cell line used to produce the polypeptide, the culture media, or during production of the polypeptide of interest (U.S. Department of Health and Human Services, Guidance for industry: Q5A Viral safety evaluation of biotechnology products derived from cell line of human or animal origin. 1998). When a polypeptide sample is applied to a Protein A column, the majority of the contaminating virus flows through the Protein A column without binding. However, some of the virus remains on the column. Surprisingly, viral clearance during protein A chromatography was increased up to 4 logs by increasing the conductivity of the elution buffer from ˜1 mS/cm to ˜6 mS/cm (Table 1). The increased elution buffer conductivity did not affect the elution of the polypeptide of interest from the Protein A column.

While not intended to be limited by any particular theory, the salt in the elution buffer may promote interactions between the virus and the protein A column (such as interactions between hydrophobic portions of the virus and the protein A column). Because of these interactions (e.g., hydrophobic interactions or other non-specific interactions), the virus may remain bound to the column longer, while the polypeptide of interest elutes from the column. If desired, the elution buffer conductivity can be optimized to further reduce the amount of virus that co-elutes with the polypeptide of interest.

TABLE 1
Viral Clearance Results and Elution Buffer Parameters

| | Antibody #1 | Antibody #2 | Antibody #3 | Antibody #4 | Antibody #5 | Antibody #6 |
|---|---|---|---|---|---|---|
| Elution | 15 mM | 15 mM | 15 mM | 25 mM | 15 mM | 25 mM |
| buffer | NaCitrate, 25 mM | NaCitrate, 25 mM | NaCitrate, | NaCitrate, | NaCitrate, 25 mM | NaCitrate, pH |
| | NaSulfate, | NaSulfate, | pH 3.2 ± 0.1 | pH 3.2 ± 0.1 | NaSulfate, | 3.2 ± 0.1 |
| | pH 3.2 ± 0.2 | pH 3.2 ± 0.2 | | | pH 3.2 ± 0.2 | |
| Conductivity, | 6 ± 2 | 6 ± 2 | 1 ± 1 | 1.5 ± 1 | 6 ± 2 | 1.5 ± 1 |
| mS/cm | | | | | | |
| Log | 4.08 | 4.87 | 2.94 | 2.68 | 5.67 | 2.02 |
| Reduction, | 4.49 | | 3.33 | 2.77 | 5.68 | 2.2 |
| XMuLV | | | 4.18 | 3.26 | | |
| | | | 3.94 | | | |
| Log | 3.43 | | 2.24 | 1.49 | 4.47 | 1.17 |
| Reduction, | 4.83 | | 2.03 | 1.63 | 4.52 | 1.47 |
| MMV | | | 3.2 | | | |

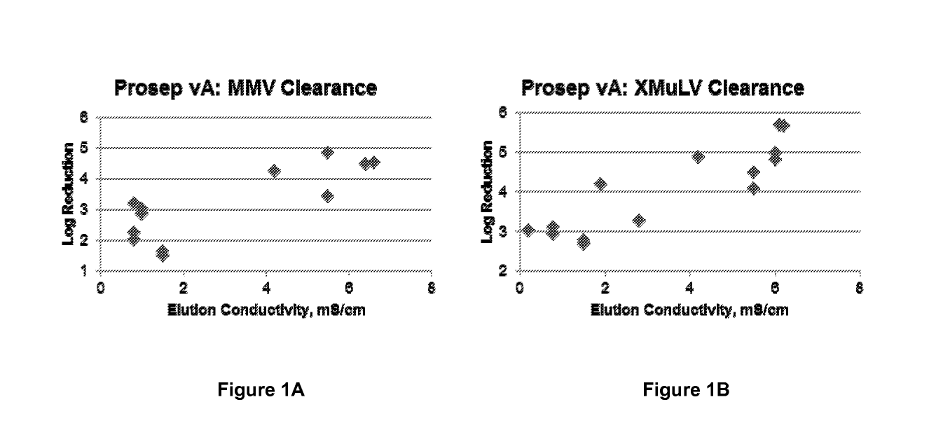

Table 1 includes the specification ranges for the conductivity values. The actual conductivity values for each experiment are included in FIGS. 1A and 1B.

### Exemplary Purification Methods

To enhance viral clearance, an elution buffer with a conductivity between about 3.0 to about 10 mS/cm can be used in standard Protein A chromatography methods (such as those described in U.S. Pat. Nos. 7,847,071 and 4,801,687; and U.S. Pub. No. 2010/0135987).

In some embodiments, the purification method involves equilibrating the Protein A resin before applying the polypeptide of interest to the resin. For example, an equilibration buffer may be applied to the Protein A resin to prepare the resin for the solution that contains the polypeptide of interest (and contaminating virus). In some embodiments, the buffer is an aqueous solution that resists changes in pH, such as weak acid and its conjugate base, or a weak base and its conjugate acid. Exemplary buffer components for an equilibration buffer include sodium phosphate, Tris, and glycine/glycinate. Exemplary concentrations of this buffer component include about 15 mM to about 300 mM, such as about any of 25, 50, 75, 100, 125, 150, 200, or 250 mM. Additionally, a salt can be included in the equilibration buffer if desired. Exemplary salts include those formed by the interaction of an acid and a base, such as sodium chloride, sodium acetate, sodium citrate, or sodium sulfate. Exemplary salt concentrations include about any of 10, 25, 50, 75, 100, 125, 150, 175, 200, 300, or 400 mM. If desired, EDTA (such as about 5 or 10 mM EDTA) may be included in the equilibration buffer. In some embodiments, the equilibration buffer has a pH between about 5.0 to about 9.0, such as about 5.1 to about 5.7 or about any of 5.1, 5.2, 5.3, 5.4, 5.5, 5.6, 5.7, 5.8, 6.0, 7.0, 8.0, or 9.0. An exemplary equilibration buffer can be found in U.S. Pat. No. 7,847,071. Another exemplary equilibration buffer is 25 mM Tris, 25 mM sodium chloride, 5 mM EDTA, pH 7.1. In some embodiments, this equilibration step is omitted.

In some embodiments, the solution that contains the polypeptide of interest (and contaminating virus) is applied to the Protein A resin under conditions such that the polypeptide of interest binds to the Protein A resin. In some embodiments, the Protein A resin is washed. In some embodiments, the equilibration buffer is used to wash the resin. In some embodiments, a wash buffer that differs from the equilibration buffer is used to wash the resin. In some embodiments, two or more different wash buffers are used. In some embodiments, the wash step elutes one or more contaminants from the resin, such as contaminates that are non-specifically bound to the resin. Preferably, the wash step does not elute a significant amount of the polypeptide of interest from the resin. Exemplary wash buffers include the equilibration buffers described above. In some embodiments, the wash buffer also includes a detergent, such as 0.1% Tween-20. In some embodiments, this wash step is omitted.

In some embodiments, the polypeptide of interest is eluted from the resin with an elution buffer having a conductivity between about 3.0 to about 10 mS/cm. In some embodiments, the elution buffer is a buffer solution that disrupts the specific interaction between an Fc region in the polypeptide of interest and the Protein A resin. In various embodiments, the conductivity of the elution buffer is between about 3 to about 3.5, about 3.5 to about 4, about 4 to about 4.5, about 4.5 to about 5, about 5 to about 5.5, about 5.5 to about 6, about 6 to about 6.5, about 6.5 to about 7, about 7 to about 7.5, about 7.5 to about 8, about 8 to about 8.5, about 8.5 to about 9, about 9 to about 9.5, or about 9.5 to about 10 mS/cm. In some embodiments, the conductivity of the elution buffer is between about 3.0 to about 10 mS/cm, such as about 3.5 to about 9.5, about 4 to about 7, or about 5 to about 6 mS/cm. In some embodiments, the conductivity of the elution buffer is about 5 or about 6 mS/cm. The conductivity of the elution buffers can be measured using standard methods, such as those described below for a Metrohm Model 712 Conductometer. In some embodiments, the pH of the elution buffer is between about 2 to about 5.5, such as about 2.5 to about 4.5, or about 3 to about 4. In some embodiments, the pH of the elution buffer is between about 2 to about 2.5, about 2.5 to about 3, about 3.0 to about 3.5, about 3.5 to about 4, about 4 to about 4.5, about 4.5 to about 5, or about 5 to about 5.5. In some embodiments, the pH of the elution buffer is about any of 3.0, 3.2, 3.5, or 4.0.

Exemplary buffer components for an elution buffer include sodium phosphate, Tris, glycine/glycinate, citrate acid, acetic acid, phosphoric acid, arginine hydrochloride, sodium citrate, glycine hydrochloride, and sodium acetate buffers. Exemplary concentrations of this buffer component include about 15 mM to about 300 mM, such as about any of 25, 50, 75, 100, 125, 150, 200, or 250 mM. In some embodiments, the elution buffer comprises citrate (e.g., sodium citrate), such as about any of 15, 20, or 25 mM citrate (e.g., sodium citrate). Additionally, a salt can be included in the elution buffer if desired. Exemplary salts include sodium chloride, sodium acetate, sodium citrate, or sodium sulfate. Exemplary salt concentrations include about any of 10, 25, 50, 75, 100, 125, 150, 175, 200, 300, or 400 mM. In some embodiments, the elution buffer comprises sulfate (e.g., sodium sulfate), such as about 50 or about 100 mM sulfate (e.g., sodium sulfate). In some embodiments, the elution buffer comprises sodium citrate and sodium sulfate.

After the polypeptide of interest is eluted from the resin, a regeneration or cleaning buffer can be used to return the Protein A resin to its original binding capacity, if desired. Exemplary regeneration/cleaning buffers include 0.1 M phosphoric acid, pH 1.5; 1% phosphoric acid; 6 M guanidine, pH 7.0; 6 M urea, pH 7.0; and 50 mM sodium hydroxide, 0.5 M sodium sulfate (Lute, S. et al., J. Chromatogr A. 26:1205(1-2):17-25, 2008).

After regeneration/cleaning, a storage buffer is optionally applied to the Protein A resin. The storage buffer remains in the resin until the next use. An exemplary storage buffer includes 100 mM sodium acetate, 2% benzyl alcohol at pH 5 or 5.2.

For these purification methods, Protein A resin is preferably incorporated into a column (Liu, H. et al., MAbs. 2(5):480-99, 2010). Alternatively, batch purification may performed, such as by adding the initial mixture to the resin in a vessel, mixing, separating the resin (for example), removing the liquid phase, washing, re-centrifuging, adding the elution buffer, re-centrifuging and removing the eluate. Sometimes a hybrid method is employed: the binding is done by the batch method, then the resin with the polypeptide of interest bound is packed onto a column and washing and elution are done on the column.

### Exemplary Protein A Resins

Any standard Protein A resin may be used in the purification methods of the present invention. Protein A is commonly used to purify polypeptides that contain an Fc region. Protein A is a 41 kDa cell surface protein from Staphylococcus aureas and binds to the Fc region of antibodies with high affinity. Protein A is stable and can be used with high salt conditions. In addition to naturally-occurring forms of Protein A, genetically modified forms of Protein A with increased stability to proteolytic degration or improved resistance to alkaline solutions are available (U.S. Pub. No. 2005/0282294). For use in affinity chromatography, the Protein A is preferably immobilized onto a solid support, such as glass, silica, agarose, or an organic polymer.

There are many commercially available Protein A resins. Examples of Protein A resin products include ProSep vA and Prosep vA Ultra by Millipore Corp.; MabSelect SuRe Protein A media and Hi-Trap rProtein-A FF from GE Healthcare; Streamline™ and MabSelect™ available from Amersham-Biosciences; Poros A and MabCapture by Applied Biosystems. Other companies that offer additional Protein A resin products include GenScript and Thermo Scientific.

### Exemplary Polypeptide of Interest

Exemplary polypeptides of interest that can be purified using the methods of the invention include any polypeptide that is capable of binding to a Protein A resin. By “polypeptide” is meant any sequence of two or more amino acids, regardless of length, post-translation modification, or function. “Protein” and “polypeptide” are used interchangeably herein. In some embodiments, the polypeptide has one or more one or more modifications, such as a post-translational modification (e.g., glycosylation, etc) or any other modification (e.g., PEGylation, etc). The polypeptide may contain one or more non-naturally-occurring amino acids (e.g., an amino acid with a side chain modification). In various embodiments, the polypeptide has at least about any of 50, 100, 150, 175, 200, 250, 300, 350, 400, or more amino acids. In some embodiments, the polypeptide includes from about 50 to about 600 amino acids, such as about 100 to about 500 amino acids, about 150 to about 400 amino acids, about 150 to about 300 amino acids, or about 175 to about 200 amino acids.

In some embodiments, the polypeptide includes a C_{H}2/C_{H}3 region that contains amino acids from the Fc region of an immunoglobulin molecule that interact with Protein A. In some embodiments, the C_{H}2/C_{H}3 region includes an intact C_{H}2 region followed by an intact C_{H}3 region. In some embodiments, the polypeptide includes an entire Fc region of an immunoglobulin. Examples of C_{H}2/C_{H}3 region region-containing polypeptides include antibodies, antibody fragments, immunoadhesins (Ashkenazi and Chamow, METHODS: A companion to Methods in Enzymology, 8:104-115, 1995) and fusion polypeptides comprising a polypeptide of interest fused to, or conjugated with, a C_{H}2/C_{H}3 region.

The term “antibody,” as used herein, refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments as well as variants (including derivatives) of antibodies and antibody fragments. Examples of molecules which are described by the term “antibody” herein include, but are not limited to, single chain Fvs (sdFvs), Fab fragments, Fab′ fragments, F(ab′)₂, disulfide linked Fvs (sdFvs), Fvs, and fragments comprising or alternatively consisting of, either a VL or a VH domain. The term “single chain Fv” or “scFv” as used herein refers to a polypeptide comprising a VH domain of antibody linked to a VL domain of an antibody. The antibodies may further comprise a heterologous polypeptide, detectable label, or other molecule.

Exemplary antibodies include, but are not limited to, monoclonal, multispecific, humanized, human or chimeric antibodies, single chain antibodies, Fab fragments, F(ab′) fragments, anti-idiotypic (anti-Id) antibodies, intracellularly-made antibodies (i.e., intrabodies), and epitope-binding fragments of any of the above. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule. In one embodiment, the immunoglobulin is an IgG1 isotype. In another embodiment, the immunoglobulin is an IgG4 isotype. Immunoglobulins may have both a heavy and light chain. An array of IgG, IgE, IgM, IgD, IgA, and IgY heavy chains may be paired with a light chain of the kappa or lambda form. In another embodiment, the antibody comprises a Fab fragment fused to a heterologous polypeptide.

The term “antibody fragment” as used herein refers to a polypeptide comprising an amino acid sequence of at least about any of 5, 10, 25, 50, 100, 150, or 200 contiguous amino acids of an antibody (including molecules such as scFvs or Fabs, that comprise, or alternatively consist of, antibody fragments or variants thereof).

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, 1gG, IgA, and IgE, respectively. See generally, Fundamental Immunology Ch. 7 (Paul, W., ed., 2nd ed. Raven Press, N.Y. (1989)). The variable regions of each light/heavy chain pair form the antibody binding site. Thus, an intact IgG antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the heavy and the light chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J Mol. Biol. 196:901-917 (1987); Chothia et al., Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al., J Immunol. 148:1547 1553 (1992). In addition, bispecific antibodies may be formed as “diabodies” (Holliger et al., “‘Diabodies’: small bivalent and bispecific antibody fragments” PNAS USA 90:6444-6448 (1993)) or “Janusins” (Traunecker et al., “Bispecific single chain molecules (Janusins) target cytotoxic lymphocytes on HIV infected cells” EMBO J 10:3655-3659 (1991) and Traunecker et al., “Janusin: new molecular design for bispecific reagents” Int J Cancer Suppl 7:51-52 (1992)).

Exemplary polypeptides of interest encompass antibodies (including antibody fragments or variants thereof), recombinantly fused or chemically conjugated (including both covalent and non-covalent conjugations) to molecules including, but not limited to, polymers, heterologous polypeptides, marker sequences, diagnostic agents and/or therapeutic agents. Additionally, exemplary polypeptides encompass antibodies (including antibody fragments or variants thereof), modified by natural processes, such as posttranslational processing, or by chemical modification techniques, which are well known in the art and discussed further herein.

In a specific embodiment, the antibody is chemically modified. This chemical modification may provide additional advantages such as increased solubility, stability and circulating time of the molecule, or decreased immunogenicity. The chemical moieties for derivitization may be selected from water soluble polymers such as polyethylene glycol, ethylene glycol/propylene glycol copolymers, carboxymethycellulose, dextran, polyvinyl alcohol and the like. The antibodies may be modified at random positions within the molecule, or at predetermined positions within the molecule and may include one, two, three, or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic polypeptide or analog). For example, the polyethylene glycol may have an average molecular weight of about any of 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999).

The polyethylene glycol molecules (or other chemical moieties) should be attached to the antibody with consideration of effects on functional or antigenic domains of the antibody. There are a number of attachment methods available to those skilled in the art, e.g., EP 0 401 384, (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to antibodies via linkage to any of a number of amino acid residues. For example, polyethylene glycol can be linked to a polypeptide via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the polypeptide or to more than one type of amino acid residue (e.g., lysine, histidine, aspartic acid, glutamic acid, cysteine and combinations thereof) of the polypeptide.

One may specifically desire antibodies chemically modified at the N-terminus. Using polyethylene glycol as an illustration, one may select from a variety of polyethylene glycol molecules (by molecular weight, branching, etc.), the proportion of polyethylene glycol molecules to polypeptide molecules in the reaction mix, the type of pegylation reaction to be performed, and the method of obtaining the selected N-terminally pegylated polypeptide. The method of obtaining the N-terminally pegylated preparation (i.e., separating this moiety from other monopegylated moieties if necessary) may be by purification of the N-terminally pegylated material from a population of pegylated polypeptide molecules. Selective polypeptides chemically modified at the N-terminus modification may be accomplished by reductive alkylation which exploits differential reactivity of different types of primary amino groups (lysine versus the N-terminal) available for derivatization in a particular polypeptide. Under the appropriate reaction conditions, substantially selective derivatization of the antibody at the N-terminus with a carbonyl group containing polymer is achieved.

As indicated above, pegylation of the antibodies may be accomplished by any number of means. For example, polyethylene glycol may be attached to the antibody either directly or by an intervening linker. Linkerless systems for attaching polyethylene glycol to polypeptides are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466.

One system for attaching polyethylene glycol directly to amino acid residues of antibodies without an intervening linker employs tresylated MPEG, which is produced by the modification of monmethoxy polyethylene glycol (MPEG) using tresylchloride (ClSO₂CH₂CF₃). Upon reaction of polypeptide with tresylated MPEG, polyethylene glycol is directly attached to amine groups of the polypeptide. In some embodiments, polypeptide-polyethylene glycol conjugates are produced by reacting antibodies with a polyethylene glycol molecule having a 2,2,2-trifluoreothane sulphonyl group.

Polyethylene glycol can also be attached to polypeptides using a number of different intervening linkers. For example, U.S. Pat. No. 5,612,460, discloses urethane linkers for connecting polyethylene glycol to polypeptides. Polypeptide-polyethylene glycol conjugates wherein the polyethylene glycol is attached to the polypeptide by a linker can also be produced by reaction of polypeptides with compounds such as MPEG-succinimidylsuccinate, MPEG activated with 1,1′-carbonyldiimidazole, MPEG-2,4,5-trichloropenylcarbonate, MPEG-p nitrophenolcarbonate, and various MPEG-succinate derivatives. A number additional polyethylene glycol derivatives and reaction chemistries for attaching polyethylene glycol to polypeptides are described in WO 98/32466.

The number of polyethylene glycol moieties optionally attached to each antibody (i.e., the degree of substitution) may also vary. For example, the pegylated antibodies may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per polypeptide molecule. Methods for determining the degree of substitution are discussed, for example, in Delgado et al., Crit. Rev. Thera. Drug Carrier Sys. 9:249-304 (1992).

Other exemplary antibodies (including antibody fragments or variants thereof) are recombinantly fused or chemically conjugated (including both covalent and non-covalent conjugations) to a heterologous polypeptide (e.g., a polypeptide unrelated to an antibody or antibody domain) or portion thereof to generate fusion polypeptides. The fusion does not necessarily need to be direct, but may occur through linker sequences. For example, antibodies may be used to target heterologous polypeptides to particular cell types (e.g., cancer cells), either in vitro or in vivo, by fusing or conjugating the heterologous polypeptides to antibodies that are specific for particular cell surface antigens or which bind antigens that bind particular cell surface receptors. Exemplary antibodies may also be fused to albumin, including but not limited to recombinant human serum albumin (see, e.g., U.S. Pat. No. 5,876,969, issued Mar. 2, 1999, EP Patent 0 413 622, and U.S. Pat. No. 5,766,883, issued Jun. 16, 1998), resulting in chimeric polypeptides. In one embodiment, antibodies (including fragments or variants thereof) are fused with polypeptide fragments comprising, or alternatively consisting of, amino acid residues of human serum albumin. In one embodiment, antibodies (including fragments or variants thereof) are fused with the mature form of human serum albumin (i.e., amino acids 1-585 of human serum albumin as shown in FIGS. 1 and 2 of EP Patent 0 322 094).

In addition, as described in U.S. Pat. No. 7,521,424, fragments of serum albumin polypeptides corresponding to an albumin polypeptide portion of an albumin fusion polypeptide, include the full length polypeptide as well as polypeptides having one or more residues deleted from the amino terminus of the amino acid sequence of the reference polypeptide (i.e., serum albumin, or a serum albumin portion of an albumin fusion polypeptide).

In addition, as described in U.S. Pat. No. 7,521,424, exemplary polypeptides include polypeptides having one or more residues deleted from the carboxy terminus of the amino acid sequence of an albumin protein corresponding to an albumin protein portion of an albumin fusion protein (e.g., serum albumin or an albumin protein portion of an albumin fusion protein).

Exemplary antibodies (including fragments or variants thereof) may be fused to either the N- or C-terminal end of a heterologous polypeptide (e.g., human serum albumin polypeptide). Heterologous polypeptides may be fused to the heavy chain or light chain constant domains of the antibodies. In one embodiment, the heterologous polypeptide is fused to the CH1 or Cκ domains. In another embodiment, the heterologous polypeptide is fused to the CH1 domain. In one embodiment, the heterologous polypeptide is from human serum albumin. Such fusion polypeptides may, for example, may increase half-life in vivo. Antibodies fused or conjugated to heterologous polypeptides may also be used in in vitro immunoassays using methods known in the art. See, e.g., PCT publication WO 93/2 1232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452 (1991).

Exemplary polypeptides further include compositions comprising, or alternatively consisting of, heterologous polypeptides fused or conjugated to antibody fragments. For example, the heterologous polypeptides may be fused or conjugated to a Fab fragment, Fd fragment, Fv fragment, F(ab)₂ fragment, or a portion thereof. Methods for fusing or conjugating polypeptides to antibody portions are known in the art. See, e.g., U.S. Pat. Nos. 5,356,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88: 10535-10539 (1991); Zheng et al., J. Immunol. 154:5590-5600 (1995); and Vil et al., Proc. Natl. Acad. Sci. USA 89:11357-11341 (1992).

Additional fusion polypeptides may be generated through the techniques of gene-shuffling, motif-shuffling, exon-shuffling, and/or codon-shuffling (collectively referred to as “DNA shuffling”). DNA shuffling may be employed to modulate the activities of antibodies (including molecules comprising, or alternatively consisting of, antibody fragments or variants thereof), such methods can be used to generate antibodies with altered activity (e.g., antibodies with higher affinities and lower dissociation rates). See, generally, U.S. Pat. Nos. 5,605,793; 5,811,238; 5,830,721; 5,834,252; and 5,837,458, and Patten et al., Curr. Opinion Biotechnol. 8:724-35 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998). In one embodiment, polynucleotides encoding antibodies may be altered by being subjected to random mutagenesis by error-prone PCR, random nucleotide insertion or other methods prior to recombination.

Exemplary polypeptides further encompass antibodies (including antibody fragments or variants thereof) conjugated to a diagnostic or therapeutic agent. The antibodies can be used diagnostically to, for example, monitor or prognose the development or progression of a tumor as part of a clinical testing procedure to, e.g., determine the efficacy of a given treatment regimen. Detection can be facilitated by coupling the antibody to a detectable substance. Examples of detectable substances include, but are not limited to, various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics. Examples of suitable enzymes include, but are not limited to, horseradish peroxidase, alkaline phosphatase, beta-galactosidase, or acetylcholinesterase; examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, but are not limited to, luciferase, luciferin, and aequorin; and examples of suitable radioactive material include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ^{113m}In, ^{115m}In), technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, and ⁹⁷Ru.

In some embodiments, an antibody (including an scFv or other molecule comprising, or alternatively consisting of, antibody fragments or variants thereof) is coupled or conjugated to a therapeutic moiety such as a cytotoxin, e.g., a cytostatic or cytocidal agent, a therapeutic agent or a radioactive metal ion, e.g., alpha-emitters such as, for example, ²¹³Bi, or other radioisotopes such as, for example ¹⁰³Pd, ¹³⁵Xe, ¹³¹I, ⁶⁸Ge, ⁵⁷Co, ⁶⁵Zn, ⁸⁵Sr, ³²P, ³⁵S, ⁹⁰Y, ¹⁵³, Sm, ¹⁵³Gd, ¹⁶⁹Yb, ⁵¹Cr, ⁵⁴Mn, ⁷⁵Se, ¹¹³Sn, ⁹⁰Y, ¹¹⁷Tin, ¹⁸⁶Re, ¹⁸⁸Re or ¹⁶⁶Ho. In specific embodiments, an antibody or fragment thereof is attached to macrocyclic chelators that chelate radiometal ions, including but not limited to, ¹⁷⁷Lu, ⁹⁰Y, ¹⁶⁶Ho, and ¹⁵³Sm, to polypeptides. In specific embodiments, the macrocyclic chelator is 1,4,7,10-tetraazacyclododecane-N,N′,N″,N′″-tetraacetic acid (DOTA). In other specific embodiments, the DOTA is attached to an antibody or fragment thereof via a linker molecule. Examples of linker molecules useful for conjugating DOTA to a polypeptide are commonly known in the art; see, for example, DeNardo et al., Clin Cancer Res. 4(10):2483-90, 1998; Peterson et al., Bioconjug. Chem. 10(4):553-7, 1999; and Zimmerman et al., Nucl. Med. Biol. 26(8):943-50, 1999.

A cytotoxin or cytotoxic agent includes any agent that is detrimental to cells. Examples include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, thymidine kinase, endonuclease, RNAse, and puromycin and fragments, variants or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Techniques known in the art may be applied to label antibodies. Such techniques include, but are not limited to, the use of bifunctional conjugating agents (see e.g., U.S. Pat. Nos. 5,756,065; 5,714,711; 5,696,239; 5,652,371; 5,505,931; 5,489,425; 5,435,990; 5,428,139; 5,342,604; 5,274,119; 4,994,560; and 5,808,003) and direct coupling reactions (e.g., Bolton-Hunter and Chloramine-T reaction).

The therapeutic agent or drug moiety is not to be construed as limited to classical chemical therapeutic agents. For example, the drug moiety may be a polypeptide possessing a desired biological activity. Such polypeptides may include, but are not limited to, for example, a toxin such as abrin, ricin A, alpha toxin, pseudomonas exotoxin, or diphtheria toxin, saporin, momordin, gelonin, pokeweed antiviral protein, alpha-sarcin and cholera toxin; a protein such as tumor necrosis factor, alpha-interferon, beta-interferon, nerve growth factor, platelet derived growth factor, tissue plasminogen activator, an apoptotic agent, e.g., TNF-alpha (TNF-α), TNF-beta, AIM I (WO 97/35899), AIM II (WO 97/34911), Fas Ligand (Takahashi et al., Int. Immunol., 6:1567-1574 (1994)), VEGI (WO 99/23105), a thrombotic agent or an anti-angiogenic agent, e.g., angiostatin or endostatin; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating a therapeutic moiety to antibodies are well known. This conjugation can be performed before or after the antibody is purified. See, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980.

Additionally, antibodies may optionally be modified by post-translational modifications including but not limited to, for example, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of procaryotic host cell expression. Modifications may include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to polypeptides such as arginylation, and ubiquitination. (See, for instance, Proteins-Structure and Molecular Properties, 2nd Ed., T. E. Creighton, W.H. Freeman and Company, New York (1993); Posttranslational Covalent Modification of Proteins, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992)). It will be appreciated that the same type of modification may be present in the same or varying degrees at several sites in a given antibody. Also, a given antibody may contain many types of modifications.

### Exemplary Methods of Producing Antibodies

Antibodies for use in the purification methods described herein can be produced using any standard method, such as any of the following antibody production methods.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of lymphoid tissues) or synthetic cDNA libraries. The DNA encoding the VH and VL domains are joined together by an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest can be selected or identified with antigen, e.g., using labeled antigen or antigen bound or captured to a solid surface or bead. Examples of phage display methods that can be used to make antibodies include, but are not limited to, those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280(1994); PCT application No. PCT/GB91/O1 134; WO 90/02809; WO 91/10737; WO 92/01047; WO 92/18719; WO 93/11236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,717; 5,780,225; 5,658,727; 5,735,743 and 5,969,108.

For some uses, such as for in vitro affinity maturation of an antibody, it may be useful to express one or more of the VH and VL domains as single chain antibodies or Fab fragments in a phage display library. For example, the cDNAs encoding the VH and VL domains may be expressed in all possible combinations using a phage display library, allowing for the selection of VH/VL combinations with preferred binding characteristics such as improved affinity or improved off rates. Additionally, VH and VL segments (such as the CDR regions of the VH and VL domains) may be mutated in vitro. Expression of VH and VL domains with “mutant” CDRs in a phage display library allows for the selection of VH/VL combinations with preferred binding characteristics such as improved affinity or improved off rates. Antibodies (including antibody fragments or variants) can be produced by any method known in the art. For example, it will be appreciated that antibodies can be expressed in cell lines other than hybridoma cell lines. Sequences encoding the cDNAs or genomic clones for the particular antibodies can be used for transformation of a suitable mammalian or nonmammalian host cells or to generate phage display libraries, for example. Additionally, antibodies may be chemically synthesized or produced through the use of recombinant expression systems.

One way to produce the antibodies would be to clone the VH and/or VL domains. In order to isolate the VH and VL domains from hybridoma cell lines, PCR primers complementary to VH or VL nucleotide sequences may be used to amplify the VH and VL sequences contained in total RNA isolated from hybridoma cell lines. The PCR products may then be cloned using vectors, for example, which have a PCR product cloning site consisting of a 5′ and 3′ single T nucleotide overhang, that is complementary to the overhanging single adenine nucleotide added onto the 5′ and 3′ end of PCR products by many DNA polymerases used for PCR reactions. The VH and VL domains can then be sequenced using conventional methods known in the art. Alternatively, the VH and VL domains may be amplified using vector specific primers designed to amplify the entire scFv, (i.e., the VH domain, linker and VL domain).

The cloned VH and VL genes may be placed into one or more suitable expression vectors. By way of non-limiting example, PCR primers including VH or VL nucleotide sequences, a restriction site, and a flanking sequence to protect the restriction site may be used to amplify the VH or VL sequences. Utilizing cloning techniques known to those of skill in the art, the PCR amplified VH domains may be cloned into vectors expressing the appropriate immunoglobulin constant region, e.g., the human IgG1 or IgG4 constant region for VH domains, and the human kappa or lambda constant regions for kappa and lambda VL domains, respectively. Preferably, the vectors for expressing the VH or VL domains comprise a promoter suitable to direct expression of the heavy and light chains in the chosen expression system, a secretion signal, a cloning site for the immunoglobulin variable domain, immunoglobulin constant domains, and a selection marker such as neomycin. The VH and VL domains may also be cloned into a single vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art (See, for example, Guo et al., J. Clin. Endocrinol. Metab. 82:925-31 (1997), and Ames et al., J. Immunol. Methods 184:177-86 (1995)).

The polynucleotides encoding antibodies may be obtained, and the nucleotide sequence of the polynucleotides determined, by any method known in the art. If the amino acid sequences of the VH domains, VL domains and CDRs thereof, are known, nucleotide sequences encoding these antibodies can be determined using methods well known in the art, i.e., the nucleotide codons known to encode the particular amino acids are assembled in such a way to generate a nucleic acid that encodes the antibody. Such a polynucleotide encoding the antibody may be assembled from chemically synthesized oligonucleotides (e.g., as described in Kutmeier et al., BioTechniques 17:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody (including molecules comprising, or alternatively consisting of, antibody fragments or variants thereof) may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells or Epstein Barr virus transformed B cell lines that express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody (including molecules comprising, or alternatively consisting of, antibody fragments or variants thereof) is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY), to generate antibodies having a different amino acid sequence, for example to create amino acid substitutions, deletions, and/or insertions.

In a specific embodiment, one or more of the VH and VL domains of heavy and light chains, or fragments or variants thereof, are inserted within antibody framework regions using recombinant DNA techniques known in the art. In a specific embodiment, one, two, three, four, five, six, or more of the CDRs of the heavy and light chains, or fragments or variants thereof, is inserted within antibody framework regions using recombinant DNA techniques known in the art. The framework regions may be naturally occurring or consensus antibody framework regions, and preferably human antibody framework regions (see, e.g., Chothia et al., J. Mol. Biol. 278: 457-479 (1998) for a listing of human antibody framework regions). Preferably, polynucleotides encoding variants of antibodies or antibody fragments having one or more amino acid substitutions may be made within the framework regions, and, preferably, the amino acid substitutions do not significantly alter binding of the antibody to its antigen. Additionally, such methods may be used to make amino acid substitutions or deletions of one or more variable region cysteine residues participating in an intrachain disulfide bond to generate antibody molecules, or antibody fragments or variants, lacking one or more intrachain disulfide bonds. Other alterations to the polynucleotide fall within the ordinary skill of the art.

In some embodiments, monoclonal antibodies are prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 571-681 (1981); Green et al., Nature Genetics 7:13-21 (1994)). Briefly, XenoMouse™ mice may be immunized with a polypeptide of interest. After immunization, the splenocytes of such mice were extracted and fused with a suitable myeloma cell line. Any suitable myeloma cell line may be employed in accordance with the present invention; however, it is preferable to employ the parent myeloma cell line (SP2O), available from the ATCC™. After fusion, the resulting hybridoma cells are selectively maintained in HAT medium, and then cloned by limiting dilution as described by Wands et al., (Gastroenterology 80:225-232 (1981)). The hybridoma cells obtained through such a selection are then assayed to identify clones which secrete the antibodies.

For some uses, including in vivo use of antibodies in humans and in vitro detection assays, it may be preferable to use human or chimeric antibodies. Completely human antibodies are particularly desirable for therapeutic treatment of human patients. In some embodiments, XenoMouse™ strains are used to produce human antibodies. See Green et al., Nature Genetics 7:13-21 (1994). See also, U.S. Pat. Nos. 4,444,887 and 4,716,111; and WO 98/46645, WO 98/50435, WO 98/24893, WO98/16654, WO 96/34096, WO 96/35735, and WO 91/10741.

A chimeric antibody is a molecule in which different portions of the antibody are derived from different immunoglobulin molecules such as antibodies having a variable region derived from a human antibody and a non-human (e.g., murine) immunoglobulin constant region or vice versa. Methods for producing chimeric antibodies are known in the art. See e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397. Chimeric antibodies comprising one or more CDRs from human species and framework regions from a non-human immunoglobulin molecule (e.g., framework regions from a murine, canine or feline immunoglobulin molecule) (or vice versa) can be produced using a variety of techniques known in the art including, for example, CDR-grafting (EP 239,400; WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,352). Often, framework residues in the framework regions are substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 352:323 (1988).

Intrabodies are antibodies, often scFvs, that are expressed from a recombinant nucleic acid molecule and engineered to be retained intracellularly (e.g., retained in the cytoplasm, endoplasmic reticulum, or periplasm). Intrabodies may be used, for example, to ablate the function of a polypeptide to which the intrabody binds. The expression of intrabodies may also be regulated through the use of inducible promoters in the nucleic acid expression vector comprising the intrabody. Exemplary intrabodies can be produced using methods known in the art, such as those disclosed and reviewed in Chen et al., Hum. Gene Ther. 5:595-601 (1994); Marasco, W. A., Gene Ther. 4:11-15 (1997); Rondon and Marasco, Annu. Rev. Microbiol. 51:257-283 (1997); Proba et al., J. Mol. Biol. 275:245-253 (1998); Cohen et al., Oncogene 17:2445-2456 (1998); Ohage and Steipe, J. Mol. Biol. 291:1119-1128 (1999); Ohage et al., J. Mol. Biol. 291:1129-1134 (1999); Wirtz and Steipe, Protein Sci. 8:2245-2250 (1999); Zhu et al., J. Immunol. Methods 231:207-222 (1999); and references cited therein.

### Exemplary Expression Systems for Producing Polypeptides of Interest

Standard expression systems can be used to produce a polypeptide of interest that can be purified using the methods of the invention. These methods typically involve construction of an expression vector(s) containing a polynucleotide that encodes the polypeptide. Vectors may include the nucleotide sequence encoding the constant region of the antibody molecule (see, e.g., WO 86/05807; WO 89/01036; and U.S. Pat. No. 5,122,464) and the variable domain of the antibody may be cloned into such a vector for expression of the entire heavy chain, the entire light chain, or both the entire heavy and light chains.

The expression vector(s) is(are) transferred to a host cell by conventional techniques and the transfected cells are then cultured by conventional techniques to produce a polypeptide of interest. In one embodiment, for the expression of antibody fragments, vectors encoding both the heavy and light chains may be co-expressed in the host cell for expression of the antibody fragment. In another embodiment, for the expression of entire antibody molecules, vectors encoding both the heavy and light chains may be co-expressed in the host cell for expression of the entire immunoglobulin molecule, as detailed below.

A variety of host-expression vector systems may be utilized to express the polypeptide of interest. Such host-expression systems represent vehicles by which the coding sequences of interest may be produced and subsequently purified, but also represent cells which may, when transformed or transfected with the appropriate nucleotide coding sequences, express the polypeptide in situ. These include, but are not limited to, bacteriophage particles engineered to express antibody fragments or variants thereof (single chain antibodies), microorganisms such as bacteria (e.g., E. coli, B. subtilis) transformed with recombinant bacteriophage DNA, plasmid DNA or cosmid DNA expression vectors containing polypeptide coding sequences; yeast (e.g., Saccharomyces, Pichia) transformed with recombinant yeast expression vectors containing polypeptide coding sequences; insect cell systems infected with recombinant virus expression vectors (e.g., baculovirus) containing polypeptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing polypeptide coding sequences; or mammalian cell systems (e.g., COS, CHO, BHK, 293, or 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, bacterial cells such as Escherichia coli, or eukaryotic cells are used for the expression of a recombinant polypeptide. For example, mammalian cells such as Chinese hamster ovary cells (CHO) in conjunction with a vector having a strong promoter are an effective expression system for polypeptides (Foecking et al., Gene 45:101 (1986); Cockett et al., Bio/Technology 8:2 (1990); Bebbington et al., Bio/Techniques 10:169 (1992); Keen and Hale, Cytotechnology 18:207 (1996)).

In bacterial systems, a number of expression vectors may be advantageously selected depending upon the use intended for the polypeptide of interest being expressed. For example, when a large quantity of such a polypeptide is to be produced, for the generation of pharmaceutical compositions of a polypeptide of interest, vectors which direct the expression of high levels of fusion polypeptide products that are readily purified may be desirable. Such vectors include, but are not limited to, the E. coli expression vector pUR278 (Ruther et al., EMBO 1. 2:1791 (1983)), in which the polypeptide coding sequence may be ligated individually into the vector in frame with the lac Z coding region so that a fusion polypeptide is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion polypeptides with glutathione 5-transferase (GST). In general, such fusion polypeptides are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) may be used as a vector to express foreign genes. The virus grows in Spodoptera frugiperda cells. Antibody coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be utilized. In cases where an adenovirus is used as an expression vector, the polypeptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) results in a recombinant virus that is viable and capable of expressing the antibody molecule in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of polypeptide products may be important for the function of the polypeptide. Different host cells have characteristic and specific mechanisms for the post-translational processing and modification of polypeptides and gene products. Appropriate cell lines or host systems can be chosen to ensure the correct modification and processing of the foreign polypeptide expressed. To this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, W138, and in particular, breast cancer cell lines such as, for example, BT483, Hs578T, HTB2, BT2O and T47D, and normal mammary gland cell line such as, for example, CRL7O3O and HsS78Bst.

For long-term, high-yield production of recombinant polypeptides, stable expression is preferred. For example, cell lines which stably express the polypeptide may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the polypeptide of interest. Such engineered cell lines may be particularly useful in screening and evaluation of compositions that interact directly or indirectly with the polypeptide of interest.

A number of selection systems may be used, including but not limited to, the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May, 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981).

The expression levels of a polypeptide of interest can be increased by vector amplification (for a review, see Bebbington and Hentschel, “The use of vectors based on gene amplification for the expression of cloned genes in mammalian cells” in DNA Cloning, Vol. 3. (Academic Press, New York, 1987)). When a marker in the vector system is amplifiable, increase in the level of inhibitor present in culture of host cell increases the number of copies of the marker gene. Since the amplified region is associated with the coding sequence of the polypeptide, production of the polypeptide will also increase (Crouse et al., Mol. Cell. Biol. 3:257 (1983)).

Once a polypeptide of interest has been recombinantly expressed, it may be purified by the method of the invention.

### EXAMPLES

The examples, which are intended to be purely exemplary of the invention and should therefore not be considered to limit the invention in any way, also describe and detail aspects and embodiments of the invention discussed above. Unless indicated otherwise, pressure is at or near atmospheric. The foregoing examples and detailed description are offered by way of illustration and not by way of limitation. All publications, patent applications, patents, journal articles, or other documents cited in this specification are herein incorporated by reference as if each individual publication, patent application, patent, journal article, or other document were specifically and individually indicated to be incorporated by reference in its entirety. In particular, all publications cited herein are expressly incorporated herein by reference for the purpose of describing and disclosing compositions and methodologies which might be used in connection with the invention. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### Example 1

### Antibody Production

The following example describes an exemplary large scale antibody production method (U.S. Pat. No. 7,064,189). One of skill in the art will be aware of routine modifications to the protocol described below.

### Cell Culture Scale-Up and Antibody Production

A serum-free and animal source-free growth medium is used from thawing cells through scale-up to the production bioreactor. The medium is stored at 2-8° C. until use.

Thawing Cells from MCB Vial(s)

Approximately 16×10⁶ cells are thawed at 37° C. in a water bath. The cells are transferred into T-225 culture flask(s) to yield approximately 50 mL working volume with an inoculation density of approximately 3.0×10⁵ cells/mL. The culture flask(s) is then placed in a humidified CO₂ incubator at 37° C. with 5% CO₂ for 4 days.

### First Expansion(s) of Culture in Spinner Flask

The culture is aseptically expanded into a 500 mL spinner flask to give approximately 300 mL working volume, at an inoculation cell density of approximately 2.2×10⁵ cells/mL. The spinner flask is then placed on magnetic stirrers in a humidified CO₂ incubator at 37° C. with 5% CO₂ for 4 days. The agitation rate for the spinner flask is 80 rpm.

The culture is again expanded aseptically into one 3000 mL spinner flask to give approximately 1500 mL working volume, at an inoculation cell density of approximately 2.2×10⁵ cells/mL. The spinner flask is then placed on magnetic stirrers in a humidified CO₂ incubator at 37° C. with 5% CO₂ for 4 days. The agitation rate for the spinner flasks is 80 rpm. If a sufficient amount of cell culture is accumulated to inoculate the seed bioreactor, proceed to the next step. If not, the culture is expanded aseptically into multiple 3000 mL spinner flasks for a total of 3 to 4 expansions, until a sufficient amount of cell culture is accumulated to inoculate the seed bioreactor.

### Seed Culture

The seed bioreactor is equipped with 2 impellers for mixing, a dissolved oxygen probe, a temperature probe, a pH probe, aseptic sampling and additional systems. The first step of the cell cultivation process is the addition of media into the bioreactor. After the media temperature reaches 37±0.5° C., the dissolved oxygen (DO) and pH levels are stabilized by addition of N₂ and CO₂ to decrease dissolved oxygen concentration to 30±5% air saturation, and obtain a pH of 7.20±0.10. The agitation rate is 80 rpm. The pooled cell culture is transferred aseptically to a 15 L seed bioreactor containing sterile growth media to yield a culture with an inoculation cell density of approximately 2.2×10⁵ cells/mL. During the cultivation process the temperature is maintained via a heat blanket and a cooling finger, the oxygen concentration is maintained via sparger and surface aeration, and pH is controlled by addition of CO₂ gas to lower the pH. The cultivation period is 5-6 days. The bioreactor air vents are protected by hydrophobic 0.2 μm vent filters.

### Production Culture

The production bioreactor is equipped with 2 impellers for mixing, 2 dissolved oxygen probes, a temperature probe, 2 pH probes, aseptic sampling and additional systems. 80 L of growth media is aseptically transferred into the 100 L production bioreactor. After the growth media temperature reaches 37±0.5° C., the DO and pH levels are stabilized by addition of N₂ and CO₂ to decrease dissolved oxygen concentration to 30±5% air saturation, and obtain a pH of 7.20±0.10. The agitation rate is 45 rpm. The 15 L seed culture is aseptically transferred into the production bioreactor to yield a culture with an inoculation cell density of approximately 2.2×10⁵ cells/mL. During the cultivation process the temperature is maintained via a heat exchanger, the oxygen concentration is maintained via sparger and surface aeration, and pH is controlled by addition of CO₂ gas to lower the pH. On day 3 after inoculation when cell density reaches approximately 1.0×10⁶ cells/mL, approximately 6 L of fed-batch media was fed into the production bioreactor. The production culture containing the antibody was harvested on Day 5 after feeding.

### Harvest of Cell Supernatant

Cell supernatant, (e.g., culture supernatant from cells expressing an antibody) is harvested on day 5 or 6 post final feeding in the final production bioreactor using a fed-batch cell culture process. The harvest process is started when the antibody concentration of at least 400 mg/L is attained. Cell culture temperature in the production bioreactor is cooled down to 15° C. at the time of harvest and maintained at that temperature during the recovery. A depth filtration process is used for cell removal and antibody recovery. The filtration process train consists of 4.5 μm, 0.45 μm and 0.2 μm pore size filters connected in series. A constant flow rate of 1.00 L/min is maintained during the operation with a cross-filter-pressure control of up to 15 psi. The 0.2 μm filtered culture supernatant is collected in a process bag and transferred for purification.

### Purification of Cell Supernatant

The supernatant can be purified using the protein A chromatography methods described herein. If desired, one or more purification steps may be performed before or after the protein A chromatography step (U.S. Pat. No. 7,064,189). For example, ion exchange, gel filtration, or hydrophobic charge interaction chromatography may be performed. Additionally, a viral inactivation step (such as incubation at low pH) may be conducted if desired (U.S. Pat. No. 7,064,189).

### Example 2

### Protein A Chromatography

The following exemplary Protein A chromatography method was used to purify polypeptides of interest. This method may be performed with any of the buffers described herein.

A Protein A based affinity column stored in storage buffer was pre-cycled with 2 column volumes (CV) of equilibration buffer, 2 CV of wash buffer, 2 CV of elution buffer, and 2 CV of regeneration buffer. The columns were then equilibrated with 4 CV of equilibration buffer (350 cm/hr).

Cell culture supernatant containing the polypeptide of interest was loaded on the Protein A based affinity column and then washed with 4 CV of equilibration buffer and then washed with 3-4 CV of wash buffer. Alternatively, the column was washed in one step with 5 CV of wash buffer. The polypeptide of interest was eluted with 3-4 CV of elution buffer and collected from OD₂₈₀. Aliquots of the pool were stored at ≦−65° C. until further analysis.

The column was stripped with 3 CV of regeneration buffer, followed by 3 CV of storage buffer. All steps were performed at 350 cm/hr. Viral clearance was measured as described in Example 4 (Table 1 and FIGS. 1A and 1B).

### Example 3

### Measuring Buffer Conductivity

The conductivity of a buffer (such as an elution buffer) may be measured using standard methods, such as those described below. Equipment used in this example included a Metrohm Model 712 Conductometer, a conductivity electrode and an immersion cell with integrated Pt100 temperature sensor (Metrohm part no. 6.0908.110). First, the meter was set up by ensuring that the main power cord was plugged into the meter and outlet, that the electrode was plugged into the proper receptacle(s) on the back of the meter, and that the power switch was on.

Next, the meter was standardized at least within 24 hours of use. In order to standardize, the “ref temp.” was set to 20° C. and the “TC const.” was set to 2.1%/° C. The electrode cell constant was noted and then the electrode was rinsed with WPU or WFI. Then the electrode was submerged into a conductivity standard; for example, a 10,000 μS/cm conductivity standard (P/N 60196). To initiate standardization, the meter was set to measurement mode, and the proper conductivity value from the conductivity standard and the standard reference temperature value were set. The cell constant calibration was performed and that reading was compared to that of the electrode cell constant reading. The standardized cell constant should be ±0.02 cm⁻¹ of the probe constant. When the standardized cell constant was out of this range, the standardization procedure was repeated and the electrode was replaced if necessary.

After the conductometer was standardized, a measurement was taken of a sample. The meter was set in measurement mode and the electrode was rinsed with either WPU or WFI. The electrode was then submerged into the sample and the value was recorded once the reading was stabilized (FIGS. 1A and 1B).

### Example 4

### Measuring Viral Clearance

All viral testing for evaluation of viral clearance was done at BioReliance according to their internal SOPs (SOP BPBT0957 and SOP OPBT0979). Alternatively, any standard method may be used to measure viral clearance, such as the methods described in Lute, S. et al., J. Chromatogr A. 26:1205(1-2):17-25, 2008 or Valdes et al., J. Biotechnology 96:251-258, 2002.

### Sample Preparation

The tests were received by BioReliance molecular Biology Laboratory. The pH of all the samples was within the range of 6-8 and therefore required no adjustments prior to extraction. Load samples were diluted 1:10, while the eluate sample was tested neat. Sample extract was prepared using the QIAamp® Viral Mini Kit as outlined in the kit procedure. The test article samples were extracted in duplicate.

The negative extraction control was prepared by extracting nuclease-free water according to kit procedure.

### PCR Amplification

Quantitative RT-PCR or PCR was performed on the samples and controls using primers and probes specific for Xenotropic Murine Leukemis Virus (X-MuLV) RNA or Murine Minute Virus (MMV) DNA with conditions optimized to achieve detection of 20 copies of XMuLV RNA (4 copies/μl) or of 50 copies of MMV DNA (10 copies/μl), respectively. Three PCR reactions were performed for each duplicate test article sample for a total of six PCR reactions per test article sample. A total of three data points for the negative test control and a total of three data points for the negative extraction control were analyzed (Table 1 and FIGS. 1A and 1B).

### Example 5

### Storage of Bulk Drug Substance

If desired, any of the following parameters may be tested for the purified polypeptide of interest. The bulk drug substance is optionally stored at 2-8° C. (short-term storage) or at or below −40° C. (long-term storage) prior to the release of the product. In-process testing of the unprocessed production bioreactor culture at harvest for each batch and in-process testing during the purification process are performed. The bioreactor is sampled aseptically and the culture is tested at various times throughout cultivation for cell density, viability and nutrient determination to ensure consistency of material being supplied for purification. The purification process is monitored at each step. Appearance is checked by visual inspection. The polypeptide concentration is determined by Absorbance at 280 nm. The pH of the material is checked. Purity is checked, for example, by SDS-PAGE and size exclusion chromatography. An ELISA may be performed to check the ability of the antibody to bind its antigen. The biological activity of the polypeptide is also monitored. Residual DNA content, Endotoxin levels, and the bioburden (the number of viable organisms present in the polypeptide preparation) are all monitored and kept at or below standard acceptable levels. Additionally the oligosaccharide content may be analyzed; the peptide sequence may also be analyzed using N-terminal sequencing and peptide mapping. Short and long-term studies of polypeptide stability may also be performed.

It is to be understood that this invention is not limited to the particular methodology, protocols, and reagents described, as these may vary. One of skill in the art will also appreciate that any methods and materials similar or equivalent to those described herein can also be used to practice or test the invention. It will be clear that the invention may be practiced otherwise than as particularly described in the foregoing description and examples. Numerous modifications and variations of the present invention are possible in light of the above teachings and, therefore, are within the scope of the appended claims.

The headings provided herein are not limitations of the various aspects or embodiments of the invention which can be had by reference to the specification as a whole.

For use herein, unless clearly indicated otherwise, use of the terms “a”, “an,” and the like refers to one or more.

Reference to “about” a value or parameter herein includes (and describes) embodiments that are directed to that value or parameter per se. For example, description referring to “about X” includes description of “X.” Numeric ranges are inclusive of the numbers defining the range.

It is understood that aspects and embodiments of the invention described herein include “comprising,” “consisting,” and “consisting essentially of” aspects and embodiments.

1. A method for separating a polypeptide of interest from a virus, the method comprising applying an elution buffer having a conductivity between about 3.5 to about 9.5 mS/cm to a Protein A resin having a polypeptide of interest and a virus adsorbed to the resin, wherein the elution of the polypeptide of interest from the resin separates the polypeptide of interest from at least a portion of the virus.

2. A method for purifying a polypeptide of interest that is capable of binding a Protein A resin, the method comprising:
a. applying a solution comprising the polypeptide of interest and a virus to the Protein A resin under conditions such that the polypeptide of interest binds to the Protein A resin;
b. washing the resin with a wash buffer; and
c. eluting the polypeptide of interest from the resin with an elution buffer having a conductivity between about 3.5 to about 9.5 mS/cm to provide a recovered composition.

3. A method, for purifying a polypeptide of interest that is capable of binding a Protein A resin, the method comprising;
a. applying a solution comprising the polypeptide of interest and a virus to the Protein A resin under conditions such that the polypeptide of interest binds to the Protein A resin;
b. washing the resin with a wash buffer;
c. eluting the polypeptide of interest from the resin with a first elution buffer to provide a recovered composition;
d. measuring the amount of virus in the recovered composition; and
e. if the amount of virus in step (d) is greater than desired, the repeating steps (a) to (c) with a second elution buffer with a higher conductivity than the first elution buffer used in step (c).

4. The method of claim 3, where steps (a) to (c) are repeated using the recovered composition.

5. The method of claim 3, where steps (a) to (c) are repeated using a solution comprising the polypeptide of interest that has not been subjected to Protein A purification.

6. The method of claim 1, wherein the conductivity of the elution buffer is between about 5 to about 6 mS/cm.

7. The method of claim 2, wherein the conductivity of the elution buffer is between about 5 to about 6 mS/cm.

8. The method of claim 3, wherein the conductivity of the second elution buffer is between about 5 to about 6 mS/cm.

9. The method of claim 1, wherein the elution buffer comprises sodium sulfate.

10. The method of claim 2, wherein the elution buffer comprises sodium sulfate.

11. The method of claim 3, wherein the second elution buffer comprises sodium sulfate.

12. The method of claim 2, wherein the amount of virus in the recovered composition is at least 10⁴-fold less than the amount of virus in the solution in step (a).

13. The method of claim 3, wherein the amount of virus in the recovered composition is at least 10⁴-fold less than the amount of virus in the solution in step (a).

14. (canceled)

15. (canceled)

16. The method of claim 1, wherein the pH of the elution buffer is between about 2.5 to about 4.

17. The method of claim 2, wherein the pH of the elution buffer is between about 2.5 to about 4.

18. The method of claim 3, wherein the pH of the second elution buffer is between about 2.5 to about 4.

19. The method of claim 1, wherein the polypeptide of interest is an antibody, antibody fragment, or a fusion polypeptide comprising an antibody or antibody fragment.

20. The method of claim 2, wherein the polypeptide of interest is an antibody, antibody fragment, or a fusion polypeptide comprising an antibody or antibody fragment.

21. The method of claim 3, wherein the polypeptide of interest is an antibody, antibody fragment, or a fusion polypeptide comprising an antibody or antibody fragment.

22-27. (canceled)
